(19) United States
(12) Patent Application Publication
(10) Pub. No.: US 2018/0353657 A1
(43) Pub. Date: Dec. 13, 2018

(54) ADHESION-PREVENTING HYDROGEL AND METHOD OF PREPARING THE SAME

(71) Applicant: SOGANG UNIVERSITY RESEARCH FOUNDATION, Seoul (KR)

(72) Inventor: Do-hyun NAM, Seoul (KR)

(21) Appl. No.: 15/775,470

(22) PCT Filed: Sep. 30, 2016
(86) PCT No.: PCT/KR2016/010995

§ 371 Date: May 11, 2018

(30) Foreign Application Priority Data
Nov. 13, 2015 (KR) 10-2015-0159510

(51) Int. Cl. A61L 31/04; A61L 31/06; A61L 31/14; A61L 31/16; C08J 3/075

(52) U.S. Cl. CPC A61L 31/042; A61L 31/041; A61L 31/06; A61L 31/145; C08J 2377/06; A61L 31/16; C08J 3/075; C08J 2305/08; A61L 31/148

(57) ABSTRACT

The present disclosure relates to an adhesion-preventing hydrogel which is formed using a biopolymer and thus is harmless to humans and has a high adhesiveness and thus achieves an excellent adhesion-preventing effect, and a method of preparing the same.

### TECHNICAL FIELD

The present disclosure relates to an adhesion-preventing hydrogel and a method of preparing the same. Particularly, the present disclosure relates to an adhesion-preventing hydrogel which is formed using a biopolymer and thus is harmless to humans and has a high adhesiveness and thus achieves an excellent adhesion-preventing effect, and a method of preparing the same.

### BACKGROUND

Adhesion occurs when an internal organ is wounded by surgery, infection, inflammation, and the like or a mesothelial basement membrane is brought into contact with surrounding tissues. Such a wound of the internal organ induces self-healing in the body and fibrin for hemostasis of the wound and tissue repair is generated through the self-healing. Such generation of fibrin is essential for self-healing of the body. However, if self-healing lasts for a long time, excessive fibrin may be produced and thus fibroblasts may be increased, resulting in the formation of new blood vessels. Therefore, the organ being self-healed may adhere to another adjacent organ.

It is generally known that in case of surgery, infection, or inflammation on an organ, the occurrence rate of adhesion of the organ reaches about 55% to about 93%. In some cases, adhesion may spontaneously dissolve, but in most cases, adhesion maintains even after wound healing, causing various sequelae. There are various sequelae caused by adhesion, and according to US statistics, examples of the sequelae may include intestinal obstruction, infertility, chronic pelvic symptom, and enterobrosia in subsequent surgery.

Accordingly, there have been a lot of studies for preventing the occurrence of adhesion by identifying the cause of adhesion during a series of processes and injecting medicine on the basis of the adhesion mechanism, and an example thereof is an anti-adhesion membrane.

The anti-adhesion membrane functions as a physical barrier to block a potential adhesion area during surgery and cover a wound of an organ in order for the wound of the organ not to be brought into contact with other surrounding tissues, and most of existing commercial anti-adhesion membranes are of solution type, film type, and gel type.

However, a solution type anti-adhesion membrane does not have a sufficient adhesiveness and thus flows down in the body and is difficult to be accurately coated on a wound, and the solution type anti-adhesion membrane may flow into another area before performing an adhesion-preventing function or may dissolve too early and thus cannot properly perform the adhesion-preventing function. Further, a film type anti-adhesion membrane has various problems such as being difficult to adhere to an internal organ and unable to be continuously and accurately positioned on a wound due to the movement of the organ.

The solution type and film type anti-adhesion membranes have a problem of being unable to properly the adhesion-preventing function since the solution type and film type anti-adhesion membranes are difficult to properly adhere to a wound of an organ. Therefore, in order to improve the adhesiveness of an anti-adhesion membrane to a wound, a gel type anti-adhesion membrane has been introduced. However, the gel type anti-adhesion membrane dissolves before the wound of the organ is healed and thus does not sufficiently stay on wounded tissues, which is a critical problem.

Accordingly, an adhesion-preventing means which can sufficiently stay on wounded tissues until a wound of an organ is healed and has a high adhesiveness to the wounded tissues of the organ and thus can provide an excellent adhesion-preventing effect desperately needs to be provided.

Korean Patent Laid-open Publication No. 2014-0115149 discloses a preparation method for hyaluronic acid, and an anti-adhesive composition comprising hyaluronic acid prepared by same preparation method.

### DISCLOSURE OF THE INVENTION

### Problems to be Solved by the Invention

The present disclosure provides an adhesion-preventing hydrogel which has a high adhesiveness to wounded tissues of an organ and thus achieves an excellent adhesion-preventing effect, and particularly provides an adhesion-preventing hydrogel including a composite of a hyaluronic acid substrate with epsilon poly-L-lysine, and the composite is prepared by heat-treating a suspension containing coagulated precipitates obtained from a mixed solution of the hyaluronic acid substrate with the epsilon poly-L-lysine.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides an adhesion-preventing hydrogel including a composite of a hyaluronic acid substrate with epsilon poly-L-lysine, and the composite is prepared by heat-treating a suspension containing coagulated precipitates obtained from a mixed solution of the hyaluronic acid substrate with the epsilon poly-L-lysine.

A second aspect of the present disclosure provides a method of preparing an adhesion-preventing hydrogel, including: preparing a mixed solution by mixing a solution containing a hyaluronic acid substrate with a solution containing epsilon poly-L-lysine; separating coagulated precipitates of the hyaluronic substrate and the epsilon poly-L-lysine formed in the mixed solution; dispersing the separated coagulated precipitates in a dispersion solvent to prepare a suspension; and heat-treating the suspension to obtain an adhesion-preventing hydrogel including a composite of the hyaluronic acid substrate with the epsilon poly-L-lysine.

### Effects of the Invention

According to an embodiment of the present disclosure, it is possible to provide an adhesion-preventing hydrogel which is excellent in adhesiveness to wounded tissues of an organ and has hydrophobic properties and also has an excellent adhesion-preventing effect due to a low flowability, and a method of preparing the same.

According to an embodiment of the present disclosure, an adhesion-preventing hydrogel including a composite of a hyaluronic acid substrate with epsilon poly-L-lysine can be prepared from a biopolymer which is harmless to humans, and, thus, it is possible to minimize a rejection of the body as a response to prevention of adhesion.

Further, according to an embodiment of the present disclosure, in the case of a narrow access route to internal organs such as minimally invasive surgery or laparoscopic surgery, the adhesion-preventing hydrogel including a composite of a hyaluronic acid substrate with epsilon poly-L-lysine can approach an organ with wounded tissues by injecting the adhesion-preventing hydrogel to the organ, and, thus, the adhesion-preventing hydrogel is applicable to various situations and easy to use.

### BRIEF DESCRIPTION OF THE DRAWINGS

FIG. 1A and FIG. 1B are a schematic diagram illustrating the generation of coagulated precipitates by stirring a mixed solution of a hyaluronic acid substrate with epsilon poly-L-lysine during a method of preparing an adhesion-preventing hydrogel in accordance with an embodiment of the present disclosure.

FIG. 2 is a schematic diagram illustrating a suspension in which coagulated precipitates are dispersed during a method of preparing an adhesion-preventing hydrogel in accordance with an embodiment of the present disclosure.

FIG. 3A to FIG. 3C are schematic diagrams illustrating the generation of a hydrogel by heat-treating a suspension in which coagulated precipitates are dispersed during a method of preparing an adhesion-preventing hydrogel in accordance with an embodiment of the present disclosure.

FIG. 4 is a schematic diagram illustrating a method of preparing an adhesion-preventing hydrogel in accordance with an embodiment of the present disclosure.

FIG. 5 is a graph showing a result of evaluation of the viscoelasticity of an adhesion-preventing hydrogel in accordance with Example 1 of the present disclosure.

FIG. 6 is a graph showing a result of evaluation of the viscoelasticity of adhesion-preventing products in accordance with Comparative Examples 1 and 2.

FIG. 7 is a graph showing a result of evaluation of the viscosity of the adhesion-preventing hydrogel in accordance with Example 1 of the present disclosure.

FIG. 8 is a graph showing a result of evaluation of the viscosity of the adhesion-preventing products in accordance with Comparative Examples 1 and 2.

FIG. 9A to FIG. 9D are images showing a result of evaluation of the flowability of an adhesion-preventing hydrogel in accordance with an example of the present disclosure.

FIG. 10A and FIG. 10B are images showing a result of evaluation of adhesion prevention of an adhesion-preventing hydrogel in accordance with an example of the present disclosure.

### BEST MODE FOR CARRYING OUT THE INVENTION

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element.

Through the whole document, the term “on” that is used to designate a position of one element with respect to another element includes both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail with reference to the accompanying drawings. However, the present disclosure may not be limited to the following embodiments, examples, and drawings.

A first aspect of the present disclosure provides an adhesion-preventing hydrogel including a composite of a hyaluronic acid substrate with epsilon poly-L-lysine, and the composite is prepared by heat-treating a suspension containing coagulated precipitates obtained from a mixed solution of the hyaluronic acid substrate with the epsilon poly-L-lysine.

In an embodiment of the present disclosure, the adhesion-preventing hydrogel is prepared from a biopolymer, and if an organ in the body is wounded by surgery or the like, the adhesion-preventing hydrogel can be used for preventing adhesion between the wounded organ and another organ by coating wounded tissues of the organ. For example, the biopolymer used as a material of the adhesion-preventing hydrogel may be a hyaluronic acid substrate and/or epsilon-poly-L-lysine.

In an embodiment of the present disclosure, the hyaluronic acid substrate may include a material, such as a hyaluronic acid or a salt of the hyaluronic acid, classified by a classification system for hyaluronic acids among various chemical materials, and for example, the hyaluronic acid substrate may include the hyaluronic acid or the salt of the hyaluronic acid.

In an embodiment of the present disclosure, the hyaluronic acid or the salt of the hyaluronic acid may be a material which can contribute to adhesion prevention of the adhesion-preventing hydrogel.

The hyaluronic acid refers to a linear polymer that contains N-acetyl glucosamine and glucuronic acid as basic units, has a molecular weight of several millions and retains the same structure in almost all biological organisms, and may be mainly a component of an extracellular matrix.

In an embodiment of the present disclosure, the extracellular matrix refers to a complicated aggregate of biopolymers filling the space inside tissues or outside cells in the body, and, thus, the hyaluronic acid which can form the extracellular matrix or the hyaluronic acid substrate which can be classified by the classification system for hyaluronic acids can be safely applied as an adhesion-preventing agent in vivo.

In an embodiment of the present disclosure, the epsilon poly-L-lysine is a material known as being safe for humans and having been used as a food preservative in the United States and Japan for years, and the epsilon poly-L-lysine is a polypeptide of L-lysine which is an essential amino acid produced via bacterial fermentation and refers to a linear polymer formed by peptide bonding between an α-carboxyl group and an ε-amino group of lysine.

Therefore, in an embodiment of the present disclosure, the adhesion-preventing hydrogel can be prepared from the hyaluronic acid substrate and the epsilon poly-L-lysine which are safe for humans, and, thus, it can be applied to and safely used in human bodies.

In an embodiment of the present disclosure, the adhesion-preventing hydrogel can be understood as a material in which a dispersed phaseis dispersed in a dispersion medium and which has low fluidity and thus does not flow well. Therefore, the adhesion-preventing hydrogel may be prepared from a biopolymer material dispersed in a dispersion medium and may be a material which has little fluidity and thus does not flow well.

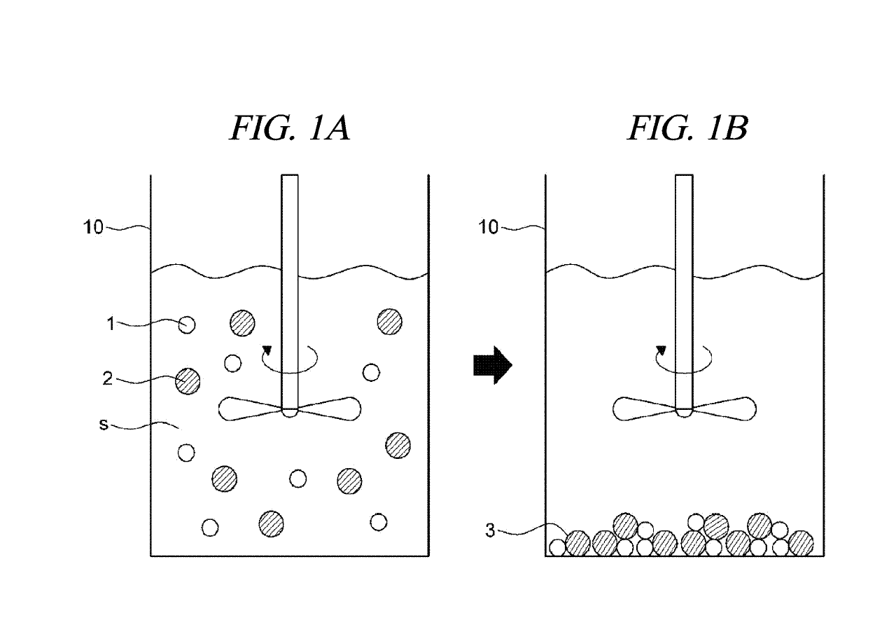

FIG. 1 illustrates the generation of coagulated precipitates by stirring a mixed solution of the hyaluronic acid substrate with the epsilon poly-L-lysine.

In an embodiment of the present disclosure, the hyaluronic acid substrate(1) may include the hyaluronic acid or the salt of the hyaluronic acid, and for example, the salt of the hyaluronic acid may be a mixture or compound of a material or two or more materials selected from the group consisting of sodium hyaluronate, potassium hyaluronate, calcium hyaluronate, magnesium hyaluronate, zinc hyaluronate, cobalt hyaluronate, tetrabutylammonium hyaluronate, and combinations thereof.

In an embodiment of the present disclosure, the hyaluronic acid substrate(1) may be an amorphous solid and the epsilon poly-L-lysine(2) may also be provided in the form of a solid. For example, each of the hyaluronic acid substrate(1) and the epsilon poly-L-lysine(2) may be provided in the form of a solution to regulate a concentration for forming the adhesion-preventing hydrogel.

In an embodiment of the present disclosure, a mixed solution of the hyaluronic acid substrate(1) with the epsilon poly-L-lysine(2) maybe prepared by mixing a solution of from about 0.1% to about 5% (w/v) hyaluronic acid substrate(1) with a solution of from about 0.1% to about 5% (w/v) epsilon poly-L-lysine(2). For example, the solution of the hyaluronic acid substrate(1) may contain from about 0.1 g to about 5 g of a hyaluronic acid or a salt of a hyaluronic acid per 100 ml of the mixed solution, and the solution of the epsilon poly-L-lysine(2) may contain from about 0.1 g to about 5 g of epsilon poly-L-lysine(2) per 100 ml of the mixed solution.

In an embodiment of the present disclosure, a weight ratio of the solution of the hyaluronic acid substrate(1) to the solution of the epsilon poly-L-lysine(2) in the mixed solution may be about 1: 0.2 to 4. For example, a weight ratio of the solution of the hyaluronic acid substrate(1) to the solution of the epsilon poly-L-lysine(2) in the mixed solution may be about 1: 0.2 to 4, about 1: 0.2 to 3, about 1: 0.2 to 2, about 1: 0.2 to 1, about 1: 1 to 4, about 1: 1 to 3, about 1:1 to 2, about 1: 2 to 4, about 1: 2 to 3, or about 1: 3 to 4.

In an embodiment of the present disclosure, when the mixed solution of the hyaluronic acid substrate(1) and the epsilon poly-L-lysine(2) is prepared, the hyaluronic acid substrate(1) and the epsilon poly-L-lysine(2) are provided as solutions to easily regulate a concentration of the mixed solution and the hyaluronic acid substrate(1) and the epsilon poly-L-lysine(2) can be mixed without a separate solvent, and, thus, the hyaluronic acid substrate(1) and the epsilon poly-L-lysine(2) can be easily mixed and reacted.

In an embodiment of the present disclosure, a molecular weight of the hyaluronic acid substrate(1) may be from about 10,000 Daltons to about 5,000,000 Daltons, and a molecular weight of the epsilon poly-L-lysine(2) may be from about 3,000 Daltons to about 10,000 Daltons.

In an embodiment of the present disclosure, for example, a molecular weight of the hyaluronic acid substrate(1) may be from about 10,000 to about 5,000,000 Daltons, from about 10,000 to about 4,000,000 Daltons, from about 10,000 to about 3,000,000 Daltons, from about 10,000 to about 2,000,000 Daltons, from about 10,000 to about 1,000,000 Daltons, from about 10,000 to about 500,000 Daltons, from about 10,000 to about 300,000 Daltons, from about 10,000 to about 100,000 Daltons, from about 10,000 to about 50,000 Daltons, from about 50,000 to about 5,000,000 Daltons, from about 50,000 to about 4,000,000 Daltons, from about 50,000 to about 3,000,000 Daltons, from about 50,000 to about 2,000,000 Daltons, from about 50,000 to about 1,000,000 Daltons, from about 50,000 to about 500,000 Daltons, from about 50,000 to about 300,000 Daltons, from about 50,000 to about 100,000 Daltons, from about 100,000 to about 5,000,000 Daltons, from about 100,000 to about 4,000,000 Daltons, from about 100,000 to about 3,000,000 Daltons, from about 100,000 to about 2,000,000 Daltons, from about 100,000 to about 1,000,000 Daltons, from about 1,000,000 to about 5,000,000 Daltons, from about 1,000,000 to about 4,000,000 Daltons, from about 100,000 to about 3,000,000 Daltons, or from about 1,000,000 to about 2,000,000 Daltons.

In an embodiment of the present disclosure, for example, a molecular weight of the epsilon poly-L-lysine(2) may be from about 3,000 to about 10,000 Daltons, from about 3,000 to about 8,000 Daltons, from about 3,000 to about 6,000 Daltons, from about 3,000 to about 4,000 Daltons, from about 4,000 to about 10,000 Daltons, from about 4,000 to about 8,000 Daltons, from about 4,000 to about 6,000 Daltons, from about 6,000 to about 10,000 Daltons, from about 6,000 to about 8,000 Daltons, from about 8,000 to about 10,000 Daltons, or from about 4,000 to about 5,000 Daltons.

In general, a polymer refers to a compound having a high molecular weight, and a molecular weight standard for a material which can be a polymer may vary depending on academic circle or theory but a material having a molecular weight of about 10,000 Daltons or more is generally referred to as a polymer compound or polymer. However, at the present time, it is useless to determine whether or not a material is a polymer on the basis of a molecular weight of the material, and according to a recent trend, the polymer may just mean having a high molecular weight.

Therefore, the hyaluronic acid substrate(1) and the epsilon poly-L-lysine(2) having a molecular weight of from about 10,000 Daltons to about 5,000,000 Daltons and a molecular weight of from about 3,000 Daltons to about 10,000 Daltons, respectively, can be considered as polymers having a high molecular weight.

In an embodiment of the present disclosure, the solution of the hyaluronic acid substrate(1) and the solution of the epsilon poly-L-lysine(2) may be mixed at the above-described weight ratio to form the mixed solution, and the hyaluronic acid substrate(1) and the epsilon poly-L-lysine(2) contained in the mixed solution may be coagulated to form the coagulated precipitates(3) by stirring the mixed solution and then may precipitate in the mixed solution.

Referring to FIG. 1, it can be seen that as the mixed solution which may contain the hyaluronic acid substrate(1), the epsilon poly-L-lysine(2), and a solvent(s) is stirred in a container (FIG. 1A), the hyaluronic acid substrate(1) and the epsilon poly-L-lysine(2) are coagulated to form the coagulated precipitates(3) and the coagulated precipitates(3) precipitate in the mixed solution (FIG. 1B).

In an embodiment of the present disclosure, the solvent(s) of the solution containing the hyaluronic acid substrate or the epsilon poly-L-Lysine may include water (distilled water), glycerol (glycerine), or propylene glycol, but may not be limited thereto.

Specifically, when the mixed solution is stirred, the hyaluronic acid substrate(1) and the epsilon poly-L-lysine(2) contained in the mixed solution coagulate with each other using intermolecular forces such as mutual attraction and thus form the coagulated precipitates(3) and precipitate to the bottom of the mixed solution.

That is, the coagulated precipitates(3) may not be a new compound produced by chemical reaction between the hyaluronic acid substrate(1) and the epsilon poly-L-lysine(2) with stirring of the mixed solution and different from the hyaluronic acid substrate(1) and the epsilon poly-L-lysine(2), but may be formed as a loosely coupled form of the hyaluronic acid substrate(1) and the epsilon poly-L-lysine(2) using intermolecular attraction as the mixed solution is stirred.

Therefore, the coagulated precipitates(3) formed as a loosely coupled form of the hyaluronic acid substrate(1) and the epsilon poly-L-lysine(2) may be added into a dispersion medium of the suspension and dispersed therein and thus may contribute to the securing of uniform properties of the hydrogel.

The coagulated precipitates(3) produced in the mixed solution and precipitated on the bottom of the mixed solution may be collected from the mixed solution and thus may contain the dispersion medium of the suspension. The coagulated precipitates(3) collected from the mixed solution can be washed several times with a dispersion solvent, and, thus, various contaminants such as non-coagulation of the hyaluronic acid substrate(1) and the epsilon poly-L-lysine(2) or impurities which may be present in the mixed solution can be separated from the coagulated precipitates(3). Therefore, the coagulated precipitates(3) with higher purity can be obtained.

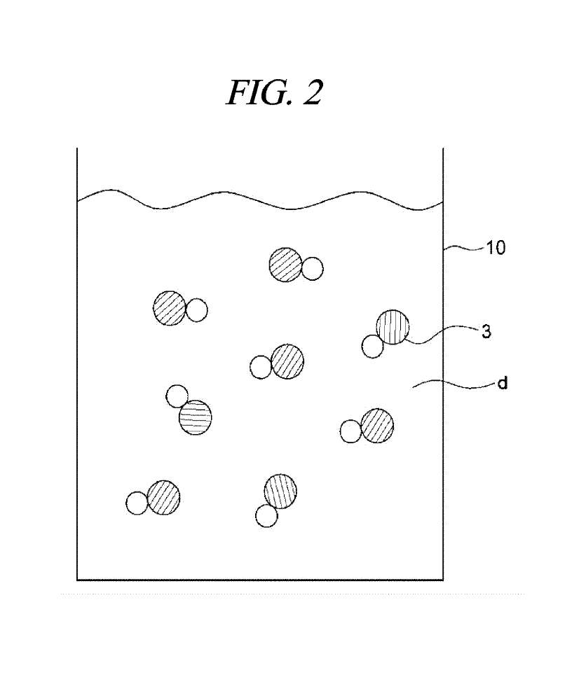

FIG. 2 illustrates a suspension in which coagulated precipitates are dispersed, in the adhesion-preventing hydrogel of the present disclosure.

The suspension may be formed by dispersing the coagulated precipitates(3) in a dispersion solvent(d). That is, the coagulated precipitates(3) may be dispersed in the dispersion solvent(d) provided as a solvent contained in the suspension, and, thus, the suspension may be heat-treated to form the hydrogel. Herein, the dispersion solvent(d) may include, for example, distilled water.

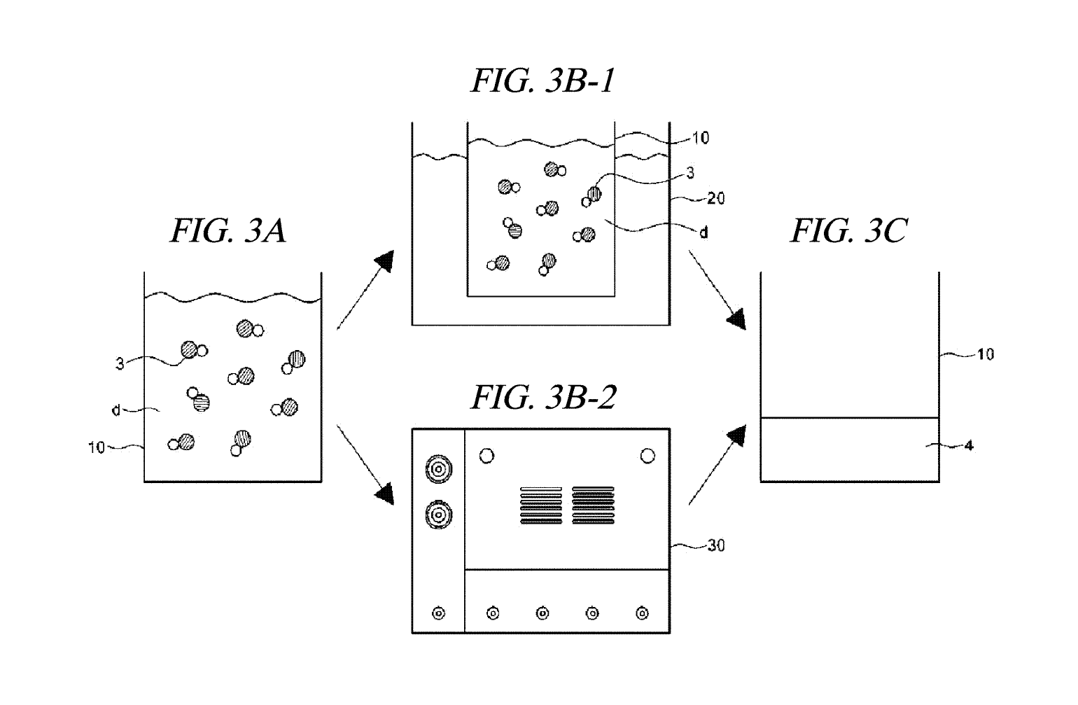

FIG. 3 shows a process of generating a hydrogel by heat-treating a suspension in which coagulated precipitates are dispersed, in the adhesion-preventing hydrogel of the present disclosure.

In an embodiment of the present disclosure, the adhesion-preventing hydrogel may be prepared by heat-treating a suspension in which coagulated precipitates in a mixed solution of the hyaluronic acid substrate and the epsilon poly-L-Lysine are dispersed.

In an embodiment of the present disclosure, the suspension may be heat-treated to form the hydrogel(4). A temperature condition for heat-treating of the suspension may be selected variously, and a heat-treating temperature and time required for heat-treating may vary depending on the means of heat-treating the suspension.

In an embodiment of the present disclosure, the adhesion-preventing hydrogel(4) may exhibit hydrophobic properties due to the heat-treating. For example, if a negative charge (− charge) of —COO in the hyaluronic acid substrate(1) reacts with a positive charge (+ charge) produced by acid salt of —NH₂ by mutual electrical attraction, the negative charge and the positive charge cancel each other out, and, thus, all the molecules of the adhesion-preventing hydrogel(4) may have hydrophobic properties and precipitate in an aqueous solution, but the present disclosure may not be limited thereto.

In an embodiment of the present disclosure, the adhesion-preventing hydrogel(4) may exhibit a high elasticity and a high adhesiveness due to the heat-treating. For example, the adhesion-preventing hydrogel may be formed as a loosely coupled form of the hyaluronic acid substrate(1) and the epsilon poly-L-lysine(2) using intermolecular attraction by stirring and heat-treating the mixed solution of the hyaluronic acid substrate(1) and the epsilon poly-L-lysine(2), and, thus, the adhesion-preventing hydrogel may exhibit a higher elasticity and a higher adhesiveness than an adhesion-preventing hydrogel which has not been heat-treated.

In an embodiment of the present disclosure, the heat-treating may be performed at a temperature ranging from about 50° C. to about 150° C. For example, the heat-treating may be performed at a temperature ranging from about 50° C. to about 150° C., from about 50° C. to about 130° C., from about 50° C. to about 110° C., from about 50° C. to about 90° C., from about 50° C. to about 70° C., from about 70° C. to about 150° C., from about 70° C. to about 130° C., from about 70° C. to about 110° C., from about 70° C. to about 90° C., from about 90° C. to about 150° C., from about 90° C. to about 130° C., from about 90° C. to about 110° C., from about 110° C. to about 150° C., from about 110° C. to about 130° C., from about 130° C. to about 150° C., or from about 70° C. to about 130° C.

In an embodiment of the present disclosure, the heat-treating of the suspension may be performed by double-boiling the suspension at a temperature ranging from about 50° C. to about 100° C. for from about 40 to about 80 minutes (FIG. 3B-1) to form the hydrogel(4). For example, the heat-treating of the suspension may be performed by double-boiling the suspension at a temperature ranging from about 50° C. to about 100° C. for from about 40 minutes to about 80 minutes, from about 40 minutes to about 70 minutes, from about 40 minutes to about 60 minutes, from about 40 minutes to about 50 minutes, from about 50 minutes to about 80 minutes, from about 50 minutes to about 70 minutes, from about 50 minutes to about 60 minutes, from about 60 minutes to about 80 minutes, from about 60 minutes to about 70 minutes, from about 70 minutes to about 80 minutes, or from about 15 minutes to about 60 minutes to form the adhesion-preventing hydrogel.

The double-boiling refers to a method of heating a heating target object not by directly heating a container(10) of the heating target object, but by placing the container(10) in a double boiler(20) containing a solvent such as water or oil and indirectly heating the heating target object, and does not require a separate complicated device for heat-treating the suspension and is economical in terms of cost of experiments. However, the double-boiling is a method of heating the suspension through a heat transfer medium such as water or oil, and, thus, the suspension can be heated by double-boiling to limited temperature.

Therefore, in the case where the suspension is heated by double-boiling, the heat-treating takes a relatively long time due to the limited temperature, and may take, for example, from about 40 minutes to about 80 minutes.

In an embodiment of the present disclosure, besides the heat-treating by double boiling, the heat-treating of the suspension may be performed using an autoclave(30) at a temperature ranging from about 100° C. to about 150° C. for from about 5 minutes to about 30 minutes (FIG. 3B-2) to form the hydrogel(4). For example, the heat-treating of the suspension may be performed using the autoclave at a temperature ranging from about 100° C. to about 150° C. for from about 5 minutes to about 30 minutes, from about 5 minutes to about 20 minutes, from about 5 minutes to about 10 minutes, from about 10 minutes to about 30 minutes, from about 10 minutes to about 20 minutes, from about 20 minutes to about 30 minutes, or from about 15 minutes to about 60 minutes to form the adhesion-preventing hydrogel.

The autoclave(30) refers to a heat-resistant pressure-resistant device capable of performing a chemical reaction, extraction, or sterilization to a heating target object under high temperature and high pressure, and in the case where the suspension is heat-treated using the autoclave(30), it may become easier to perform heat-treating to the suspension due to a pressure resistant function provided by the autoclave(30). Therefore, the heat-treating using the autoclave(30) requires a shorter time and a higher temperature to form the hydrogel(4) than the heat-treating by double-boiling.

In an embodiment of the present disclosure, if the heat-treating of the suspension is performed for less than 40 minutes, the adhesion-preventing hydrogel may not be completely formed and may coexist with opaque white precipitates. If the heat-treating of the suspension is performed for more than 80 minutes, heating may continue even after the adhesion-preventing hydrogel is completely formed, and, thus, a part of the polymer of the formed adhesion-preventing hydrogel may be decomposed and depolymerized. Therefore, desirably, the heat-treating of the suspension may be performed for from about 40 minutes to about 80 minutes.

In an embodiment of the present disclosure, the heat-treating of the suspension includes the heat-treating by double-boiling or the heat-treating using the autoclave(30), but may include any other heat-treating means as long as it can form the adhesion-preventing hydrogel(4) of the present disclosure from the suspension.

The purpose of the heat-treating is to form the hydrogel(4) having adhesiveness by using the coagulated precipitates(3) of the hyaluronic acid substrate(1) and the epsilon poly-L-lysine(2).

However, the adhesion-preventing hydrogel(4) of the present disclosure is provided to prevent adhesion in a human body and more specifically adhesion between organs of the human body. To this end, sterilization of the hydrogel(4) is essential. The adhesion-preventing hydrogel(4) can be sterilized through the heat-treating of the suspension, and, thus, even if it is applied to a human for preventing adhesion, its safety can be secured.

A second aspect of the present disclosure provides a method of preparing an adhesion-preventing hydrogel, including: preparing a mixed solution by mixing a solution containing a hyaluronic acid substrate with a solution containing epsilon poly-L-lysine; separating coagulated precipitates of the hyaluronic substrate and the epsilon poly-L-lysine formed in the mixed solution; dispersing the separated coagulated precipitates in a dispersion solvent to prepare a suspension; and heat-treating the suspension to obtain an adhesion-preventing hydrogel including a composite of the hyaluronic acid substrate with the epsilon poly-L-lysine.

Detailed descriptions of the method of preparing an adhesion-preventing hydrogel according to the second aspect of the present disclosure, which overlap with those of the first aspect of the present disclosure, are omitted hereinafter, but the descriptions of the first aspect of the present disclosure may be identically applied to the second aspect of the present disclosure, even though they are omitted hereinafter.

In an embodiment of the present disclosure, the adhesion-preventing hydrogel is prepared from a biopolymer, and if an organ in the body is wounded by surgery or the like, the adhesion-preventing hydrogel can be used for preventing adhesion between the wounded organ and another organ by coating wounded tissues of the organ. For example, the biopolymer used as a material of the adhesion-preventing hydrogel may be a hyaluronic acid substrate and/or epsilon-poly-L-lysine.

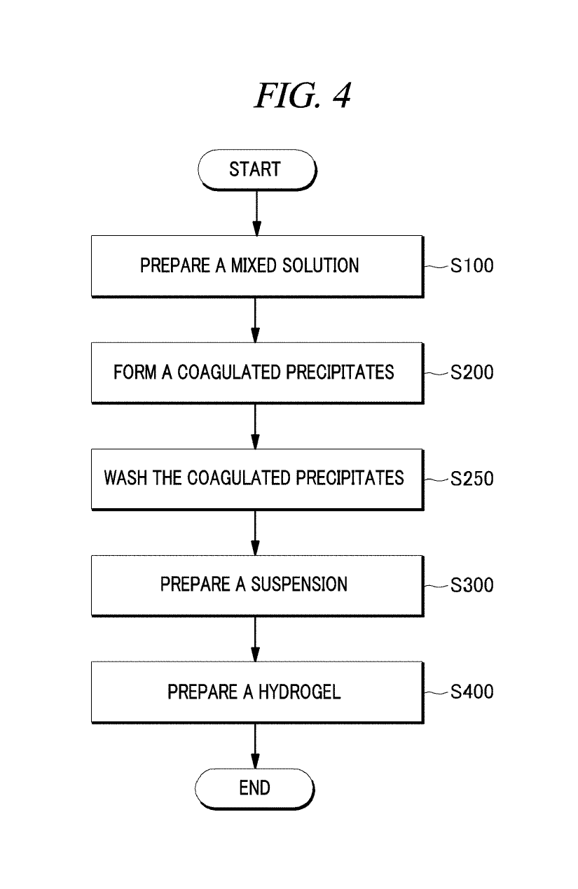

In an embodiment of the present disclosure, the method of preparing an adhesion-preventing hydrogel can be explained using a flowchart in FIG. 4.

In an embodiment of the present disclosure, the method of preparing an adhesion-preventing hydrogel may include a mixed solution preparing process (S100) for preparing the mixed solution by mixing a solution of the hyaluronic acid substrate(1) with a solution of the epsilon poly-L-lysine(2), a coagulated precipitate forming process (S200) for forming the coagulated precipitates(3) by stirring the mixed solution and coagulating the hyaluronic acid substrate(1) and the epsilon poly-L-lysine(2), a suspension preparing process (S300) for preparing a suspension by dispersing the coagulated precipitates(3) collected from the mixed solution in the dispersion solvent(d), and a hydrogel preparing process (S400) for preparing the hydrogel(4) by heat-treating the suspension.

In an embodiment of the present disclosure, the dispersion solvent may include water (distilled water), glycerol (glycerine), or propylene glycol, but may not be limited thereto.

In the mixed solution preparing process (S100), a weight ratio of the solution of the hyaluronic acid substrate(1) to the solution of the epsilon poly-L-lysine(2) may be about 1: 0.2 to 4. For example, a weight ratio of the solution of the hyaluronic acid substrate(1) to the solution of the epsilon poly-L-lysine(2) in the mixed solution may be about 1: 0.2 to 4, about 1: 0.2 to 3, about 1: 0.2 to 2, about 1: 0.2 to 1, about 1: 1 to 4, about 1: 1 to 3, about 1: 1 to 2, about 1: 2 to 4, about 1: 2 to 3, or about 1: 3 to 4.

In an embodiment of the present disclosure, the solvent of the solution containing the hyaluronic acid substrate or the epsilon poly-L-Lysine may include water (distilled water), glycerol (glycerine), or propylene glycol, but may not be limited thereto.

In an embodiment of the present disclosure, the hyaluronic acid substrate may include the hyaluronic acid or the salt of the hyaluronic acid, and for example, the salt of the hyaluronic acid may be a mixture or compound of a material or two or more materials selected from the group consisting of sodium hyaluronate, potassium hyaluronate, calcium hyaluronate, magnesium hyaluronate, zinc hyaluronate, cobalt hyaluronate, tetrabutylammonium hyaluronate, and combinations thereof.

The hyaluronic acid refers to a linear polymer that contains N-acetyl glucosamine and glucuronic acid as basic units, has a molecular weight of several millions and retains the same structure in almost all biological organisms, and may be mainly a component of an extracellular matrix.

In an embodiment of the present disclosure, the extracellular matrix refers to a complicated aggregate of biopolymers filling the space inside tissues or outside cells in the body, and, thus, the hyaluronic acid which can form the extracellular matrix or the hyaluronic acid substrate which can be classified by the classification system for hyaluronic acids can be safely applied as an adhesion-preventing agent in vivo.

In an embodiment of the present disclosure, the epsilon poly-L-lysine is a material known as being safe for humans and having been used as a food preservative in the United States and Japan for years, and the epsilon poly-L-lysine is a polypeptide of L-lysine which is an essential amino acid produced via bacterial fermentation and refers to a linear polymer formed by peptide bonding between an α-carboxyl group and an ε-amino group of lysine.

Therefore, in an embodiment of the present disclosure, the adhesion-preventing hydrogel can be prepared from the hyaluronic acid substrate and the epsilon poly-L-lysine which are safe for humans, and, thus, it can be applied to and safely used in human bodies.

In an embodiment of the present disclosure, the adhesion-preventing hydrogel can be understood as a material in which a dispersed phase is dispersed in a dispersion medium and which has low fluidity and thus does not flow well. Therefore, the adhesion-preventing hydrogel may be prepared from a biopolymer material dispersed in a dispersion medium and may be a material which has little fluidity and thus does not flow well.

In an embodiment of the present disclosure, a molecular weight of the hyaluronic acid or the salt of the hyaluronic acid may be from about 10,000 Daltons to about 5,000,000 Daltons, and a molecular weight of the epsilon poly-L-lysine(2) may be from about 3,000 Daltons to about 10,000 Daltons.

In an embodiment of the present disclosure, for example, a molecular weight of the hyaluronic acid or the salt of the hyaluronic acid may be from about 10,000 to about 5,000,000 Daltons, from about 10,000 to about 4,000,000 Daltons, from about 10,000 to about 3,000,000 Daltons, from about 10,000 to about 2,000,000 Daltons, from about 10,000 to about 1,000,000 Daltons, from about 10,000 to about 500,000 Daltons, from about 10,000 to about 300,000 Daltons, from about 10,000 to about 100,000 Daltons, from about 10,000 to about 50,000 Daltons, from about 50,000 to about 5,000,000 Daltons, from about 50,000 to about 4,000,000 Daltons, from about 50,000 to about 3,000,000 Daltons, from about 50,000 to about 2,000,000 Daltons, from about 50,000 to about 1,000,000 Daltons, from about 50,000 to about 500,000 Daltons, from about 50,000 to about 300,000 Daltons, from about 50,000 to about 100,000 Daltons, from about 100,000 to about 5,000,000 Daltons, from about 100,000 to about 4,000,000 Daltons, from about 100,000 to about 3,000,000 Daltons, from about 100,000 to about 2,000,000 Daltons, from about 100,000 to about 1,000,000 Daltons, from about 1,000,000 to about 5,000,000 Daltons, from about 1,000,000 to about 4,000,000 Daltons, from about 100,000 to about 3,000,000 Daltons, or from about 1,000,000 to about 2,000,000 Daltons.

In an embodiment of the present disclosure, for example, a molecular weight of the epsilon poly-L-lysine(2) may be from about 3,000 to about 10,000 Daltons, from about 3,000 to about 8,000 Daltons, from about 3,000 to about 6,000 Daltons, from about 3,000 to about 4,000 Daltons, from about 4,000 to about 10,000 Daltons, from about 4,000 to about 8,000 Daltons, from about 4,000 to about 6,000 Daltons, from about 6,000 to about 10,000 Daltons, from about 6,000 to about 8,000 Daltons, from about 8,000 to about 10,000 Daltons, or from about 4,000 to about 5,000 Daltons.

In general, a polymer refers to a compound having a high molecular weight, and a molecular weight standard for a material which can be a polymer may vary depending on academic circle or theory but a material having a molecular weight of about 10,000 Daltons or more is generally referred to as a polymer compound or polymer. However, at the present time, it is useless to determine whether or not a material is a polymer on the basis of a molecular weight of the material, and according to a recent trend, the polymer may just mean having a high molecular weight.

Therefore, the hyaluronic acid substrate(1) and the epsilon poly-L-lysine(2) having a molecular weight of from about 10,000 Daltons to about 5,000,000 Daltons and a molecular weight of from about 3,000 Daltons to about 10,000 Daltons, respectively, can be considered as polymers having a high molecular weight.

For example, the hyaluronic acid substrate(1) may have a higher molecular weight and a wider range of molecular weight than the epsilon poly-L-lysine(2), and the hyaluronic acid which is conventionally available in the market and provides a classification system of the hyaluronic acid substrate(1) may have properties that may vary depending on its molecular weight. However, the adhesion-preventing hydrogel(4) is not greatly affected by a molecular weight of the hyaluronic acid substrate(1) contained in the adhesion-preventing hydrogel(4) and can provide an adhesion-preventing effect as a feature of the adhesion-preventing hydrogel(4) which is the purpose of the present disclosure.

In an embodiment of the present disclosure, in the coagulated precipitate forming process (S200) after the mixed solution preparing process (S100), the mixed solution prepared by the mixed solution preparing process (S100) may be stirred to form the coagulated precipitates(3) as coagulation of the hyaluronic acid substrate(1) and the epsilon poly-L-lysine(2).

Specifically, when the mixed solution is stirred, the hyaluronic acid substrate and the epsilon poly-L-Lysine contained in the mixed solution coagulate with each other using intermolecular forces such as mutual attraction and thus form the coagulated precipitates and precipitate to the bottom of the mixed solution.

That is, the coagulated precipitates may not be a new compound produced by chemical reaction between the hyaluronic acid substrate and the epsilon poly-L-Lysine with stirring of the mixed solution and different from the hyaluronic acid substrate and the epsilon poly-L-Lysine, but may be formed as a loosely coupled form of the hyaluronic acid substrate and the epsilon poly-L-Lysine using intermolecular attraction as the mixed solution is stirred.

Therefore, the coagulated precipitates formed as a loosely coupled form of the hyaluronic acid substrate and the epsilon poly-L-Lysine may be added into a dispersion medium of the suspension and dispersed therein and thus may contribute to the securing of uniform properties of the hydrogel.

The coagulated precipitates produced in the mixed solution and precipitated on the bottom of the mixed solution may be collected from the mixed solution and thus may contain the dispersion medium of the suspension. The coagulated precipitates collected from the mixed solution can be washed several times with a dispersion solvent, and, thus, various contaminants such as non-coagulation of the hyaluronic acid substrate and the epsilon poly-L-Lysine or impurities which may be present in the mixed solution can be separated from the coagulated precipitates. Therefore, the coagulated precipitates with higher purity can be obtained. Herein, the dispersion solvent(d) may include, for example, distilled water.

In an embodiment of the present disclosure, the coagulated precipitates may be formed by stirring the mixed solution for from 20 minutes to 30 minutes to coagulate the hyaluronic acid substrate and the epsilon poly-L-Lysine and then precipitating the coagulation in the mixed solution. For example, in the coagulated precipitate forming process (S200), the mixed solution may be stirred for from about 20 minutes to about 30 minutes. If a negative charge (− charge) of —COO in the hyaluronic acid substrate(1) reacts with a positive charge (+ charge) produced by acid salt of —NH₂ by mutual electrical attraction as the mixed solution is stirred, the negative charge and the positive charge cancel each other out, and, thus, all the molecules of the adhesion-preventing hydrogel(4) may have hydrophobic properties, and, thus, the hyaluronic acid substrate(1) and the epsilon poly-L-lysine(2) contained in the mixed solution may form the coagulated precipitates(3) due to a difference in solubility and precipitate in the mixed solution.

In an embodiment of the present disclosure, the method may further include washing the coagulated precipitates multiple times with distilled water and then dispersing the coagulated precipitates in the dispersion solvent. For example, the coagulated precipitates(3) formed in the coagulated precipitate forming process (S200) may be collected from the mixed solution and thus may contain the dispersion medium of the suspension, and before that, the coagulated precipitates(3) may be washed multiple times with distilled water (S250).

Therefore, impurities which may be present between the coagulated precipitates(3) provided as precipitated in the mixed solution can be removed from the coagulated precipitates(3) through the washing with the distilled water, and the hyaluronic acid substrate(1) can be a component of the hydrogel(4) applicable to organs of a human body and prevented from contamination with a higher purity by using the coagulated precipitates(3) from which contaminants have been previously removed.

Then, in the suspension preparing process (S300), the coagulated precipitates(3) may be dispersed in the dispersion solvent(d) to form the suspension.

In an embodiment of the present disclosure, the suspension refers to a liquid in which fine solid particles are dispersed and floated, and may be a mixture of a solute, which is provided as the coagulated precipitates(3) of the hyaluronic acid substrate(1) and the epsilon poly-L-lysine(2), dispersed in a solvent, which is provided as the dispersion solvent(d) such as distilled water.

Then, the suspension prepared in the suspension preparing process (S300) may turn into the hydrogel(4) through the hydrogel preparing process (S400).

In an embodiment of the present disclosure, in the hydrogel preparing process (S400), the adhesion-preventing hydrogel may be prepared by heat-treating the suspension at a temperature ranging from about 50° C. to about 100° C. For example, the adhesion-preventing hydrogel may be formed as a loosely coupled form of the hyaluronic acid substrate(1) and the epsilon poly-L-lysine(2) using intermolecular attraction by heat-treating the suspension of the mixed solution of the hyaluronic acid substrate(1) and the epsilon poly-L-lysine(2), and, thus, the adhesion-preventing hydrogel may exhibit a higher elasticity and a higher adhesiveness than an adhesion-preventing hydrogel which has not been heat-treated. A temperature condition for heat-treating of the suspension may be selected variously, and a heat-treating temperature and time required for heat-treating may vary depending on the means of heat-treating the suspension.

In an embodiment of the present disclosure, in the hydrogel preparing process (S400), the hydrogel(4) may be prepared by double-boiling the suspension at a temperature ranging from about 50° C. to about 100° C. for from about 40 minutes to about 80 minutes or heat-treating the suspension using the autoclave(30) at a temperature ranging from about 100° C. to about 150° C. for from about 5 minutes to about 30 minutes.

In an embodiment of the present disclosure, for example, the heat-treating of the suspension may be performed by double-boiling the suspension at a temperature ranging from about 50° C. to about 100° C. for from about 40 minutes to about 80 minutes, from about 40 minutes to about 70 minutes, from about 40 minutes to about 60 minutes, from about 40 minutes to about 50 minutes, from about 50 minutes to about 80 minutes, from about 50 minutes to about 70 minutes, from about 50 minutes to about 60 minutes, from about 60 minutes to about 80 minutes, from about 60 minutes to about 70 minutes, from about 70 minutes to about 80 minutes, or from about 15 minutes to about 60 minutes to form the adhesion-preventing hydrogel.

The double-boiling refers to a method of heating a heating target object not by directly heating a container(10) of the heating target object, but by placing the container(10) in a double boiler(20) containing a solvent such as water or oil and indirectly heating the heating target object, and does not require a separate complicated device for heat-treating the suspension and is economical in terms of cost of experiments. However, the double-boiling is a method of heating the suspension through a heat transfer medium such as water or oil, and, thus, the suspension can be heated by double-boiling to limited temperature.

Therefore, in the case where the suspension is heated by double-boiling, the heat-treating takes a relatively long time due to the limited temperature, and may take, for example, from about 40 minutes to about 80 minutes.

In an embodiment of the present disclosure, for example, the heat-treating of the suspension may be performed using the autoclave at a temperature ranging from about 100° C. to about 150° C. for from about 5 minutes to about 30 minutes, from about 5 minutes to about 20 minutes, from about 5 minutes to about 10 minutes, from about 10 minutes to about 30 minutes, from about 10 minutes to about 20 minutes, from about 20 minutes to about 30 minutes, or from about 15 minutes to about 60 minutes to form the adhesion-preventing hydrogel.

The autoclave(30) refers to a heat-resistant pressure-resistant device capable of performing a chemical reaction, extraction, or sterilization to a heating target object under high temperature and high pressure, and in the case where the suspension is heat-treated using the autoclave(30), it may become easier to perform heat-treating to the suspension due to a pressure resistant function provided by the autoclave(30). Therefore, the heat-treating using the autoclave(30) requires a shorter time and a higher temperature to form the hydrogel(4) than the heat-treating by double-boiling.

In an embodiment of the present disclosure, if the heat-treating of the suspension is performed for less than 40 minutes, the adhesion-preventing hydrogel may not be completely formed and may coexist with opaque white precipitates. If the heat-treating of the suspension is performed for more than 80 minutes, heating may continue even after the adhesion-preventing hydrogel is completely formed, and, thus, a part of the polymer of the formed adhesion-preventing hydrogel may be decomposed and depolymerized. Therefore, desirably, the heat-treating of the suspension may be performed for from about 40 minutes to about 80 minutes.

The heat-treating by double-boiling and the heat-treating using the autoclave(30) can be selectively used by an operator who can prepare the hydrogel(4) by applying the method of preparing an adhesion-preventing hydrogel.

In an embodiment of the present disclosure, the heat-treating of the suspension includes the heat-treating by double-boiling or the heat-treating using the autoclave(30), but may include any other heat-treating means as long as it can form the adhesion-preventing hydrogel(4) of the present disclosure from the suspension.

In an embodiment of the present disclosure, the adhesion-preventing hydrogel(4) is provided to prevent adhesion in a human body and more specifically adhesion between organs of the human body. To this end, sterilization of the hydrogel(4) is essential. The adhesion-preventing hydrogel(4) can be sterilized through the heat-treating of the suspension, and, thus, even if it is applied to a human for preventing adhesion, its safety can be secured.

### MODE FOR CARRYING OUT THE INVENTION

Hereinafter, the present disclosure will be explained in more detail with reference to Examples. However, the following Examples are illustrative only for better understanding of the present disclosure but do not limit the present disclosure.

### Preparation of Adhesion-Preventing Hydrogel

Adhesion-preventing hydrogels were classified into Examples 1 to 4 depending on a molecular weight of the sodium hyaluronate, a means of heat-treating the suspension, a heat-treating temperature for the suspension, and time required for heat-treating the suspension, as shown in the following Table 1. Further, adhesion-preventing agents according to Comparative Examples 1 to 3 were selected from products of a first company, a second company, and a third company and prepared as control groups of the adhesion-preventing hydrogel of Example 1 (Comparative Example 1: Medicurtain, Comparative Example 2: Guardix-sol, and Comparative Example 3: Protescal).

### Example 1

First, a mixed solution was prepared by mixing a solution of 0.5% (w/v) sodium hyaluronate having a molecular weight of 2,000,000 Daltons and a solution of 0.5% (w/v) epsilon poly-L-lysine having a molecular weight of 4,500 at a weight ratio of 1:1, and each solvent in the prepared mixed solution was distilled water. The prepared mixed solution was stirred in a container for 30 minutes to form coagulated precipitates in the mixed solution. The coagulated precipitates were collected from the mixed solution and washed three times with distilled water and then dispersed in distilled water serving as a dispersion solvent. Finally, a suspension in which the coagulated precipitates were dispersed in the dispersion solvent was heat-treated using an autoclave at 124° C. for 15 minutes to obtain an adhesion-preventing hydrogel.

### Example 2

An adhesion-preventing hydrogel was prepared in the same manner as in Example 1 except that a suspension dispersed in distilled water was heat-treated by double-boiling at 80° C. for 60 minutes to obtain an adhesion-preventing hydrogel.

### Example 3

A mixed solution was prepared by mixing a solution of 0.5% (w/v) sodium hyaluronate having a molecular weight of 1,000,000 Daltons and a solution of 0.5% (w/v) epsilon poly-L-lysine having a molecular weight of 4,500 at a weight ratio of 1:1. The prepared mixed solution was stirred in a container for 30 minutes to form coagulated precipitates in the mixed solution. The coagulated precipitates were collected from the mixed solution and washed three times with distilled water and then dispersed in distilled water. Finally, a suspension in which the coagulated precipitates were dispersed in the distilled water was heat-treated using an autoclave at 124° C. for 15 minutes to obtain an adhesion-preventing hydrogel.

### Example 4

An adhesion-preventing hydrogel was prepared in the same manner as in Example 3 except that a suspension dispersed in distilled water was heat-treated by double-boiling at 80° C. for 60 minutes to obtain an adhesion-preventing hydrogel.

TABLE 1

| | | Molecular | | | Time (min) |
|---|---|---|---|---|---|
| | Molecular weight | weight (Dalton) | Means of | Heat-treating | required for |
| | (Dalton) of sodium | of epsilon poly- | heat-treating | temperature (° C.) | heat-treating |
| | hyaluronate | L-lysine | suspension | for suspension | suspension |
| Example 1 | 2,000,000 | 4,500 | Autoclave | 124 | 15 |
| Example 2 | 2,000,000 | 4,500 | Double-boiling | 80 | 60 |
| Example 3 | 1,000,000 | 4,500 | Autoclave | 124 | 15 |
| Example 4 | 1,000,000 | 4,500 | Double-boiling | 80 | 60 |

### Evaluation of Properties of Adhesion-Preventing Hydrogel

### 1. Rheological Property of Adhesion-Preventing Hydrogel

The viscoelasticity and viscosity of the adhesion-preventing hydrogel according to Example 1 was evaluated using an AR200ex rheometer. The viscoelasticity and viscosity of Medicurtain (hyaluronic acid+hydroxyethyl starch, Comparative Example 1) and Guardix-sol (hyaluronic acid+carboxymethyl cellulose, Comparative Example 2) as Comparative Examples was evaluated using an AR200ex rheometer and then shown in the following Table 2 together with the evaluation result of Example 1.

TABLE 2

| | G′/G″ | Viscosity |
|---|---|---|
| | (Pa)-3.5 Hz | (Pa · s) |
| Example 1 (HA + PLL) | 14,000/12,000 | 2,300 |
| Comparative Example 1 (HA + HES) | 119.9/52 | 69 |
| Medicurtain (Shinpoong Pharm. Co., Ltd.) | | |
| Comparative Example 2 (HA + CMC) | 3.94/10.65 | 1.13 |
| Guardix (Hanmi Pharm. Co., Ltd.) | | |

*HA = Hyaluronic Acid,
*HES = HydroxyEthyl Starch,
*CMC = CarboxyMethyl Cellulose

As shown in Table 2, it can be seen that the adhesion-preventing hydrogel of the present disclosure exhibits a higher viscoelasticity and a higher viscosity than the products of other companies according to Comparative Examples 1 and 2.

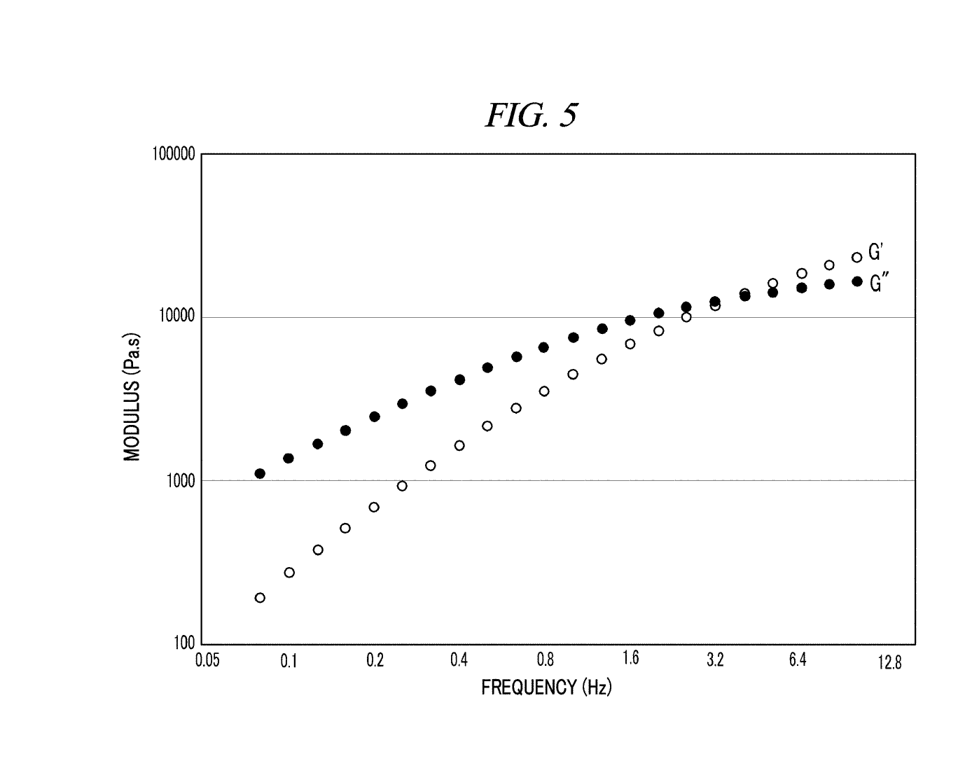

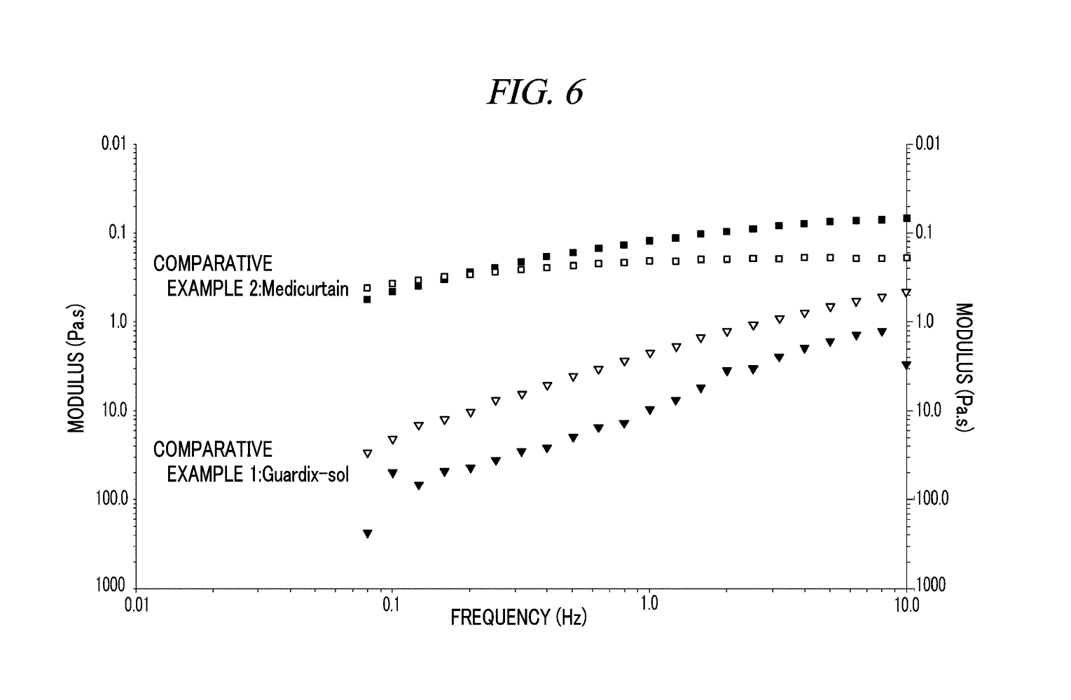

FIG. 5 shows a result of evaluation of the viscoelasticity of an adhesion-preventing hydrogel in accordance with Example 1 of the present disclosure, and FIG. 6 shows a result of evaluation of the viscoelasticity of adhesion-preventing hydrogels as products of other companies (Comparative Example 1: Medicurtain, Comparative Example 2: Guardix-sol). As shown in FIG. 5, it can be seen that as the number of vibrations increases, a storage modulus G′ which is the amount of elastic energy accumulated in the vibrating adhesion-preventing hydrogel according to Example 1 and a loss modulus G″ which is the amount of elastic energy lost by the vibrating hydrogel in each vibration have an intersection. The intersection between the storage modulus G′ and the loss modulus G″ is a typical property of a liquid polymer having elasticity, and it can be seen from the result of evaluation of the viscoelasticity that the adhesion-preventing hydrogel according to Example 1 has elasticity. Herein, the adhesion-preventing hydrogel according to Example 1 was formed not by cross-linking of a cross-linking agent to give high elasticity but by heating a mixture of sodium hyaluronate and epsilon poly-L-lysine, and it was confirmed that the adhesion-preventing hydrogel according to Example 1 has a storage modulus G′ close to 10,000 Pa. Accordingly, it was confirmed that the adhesion-preventing hydrogel according to Example 1 has a storage modulus G′ much higher than a typical storage modulus G′, i.e., 500 Pa to 1,200 Pa, of the existing commercial cross-linked hydrogel products for adhesion prevention according to Comparative Examples 1 and 2 as shown in FIG. 6.

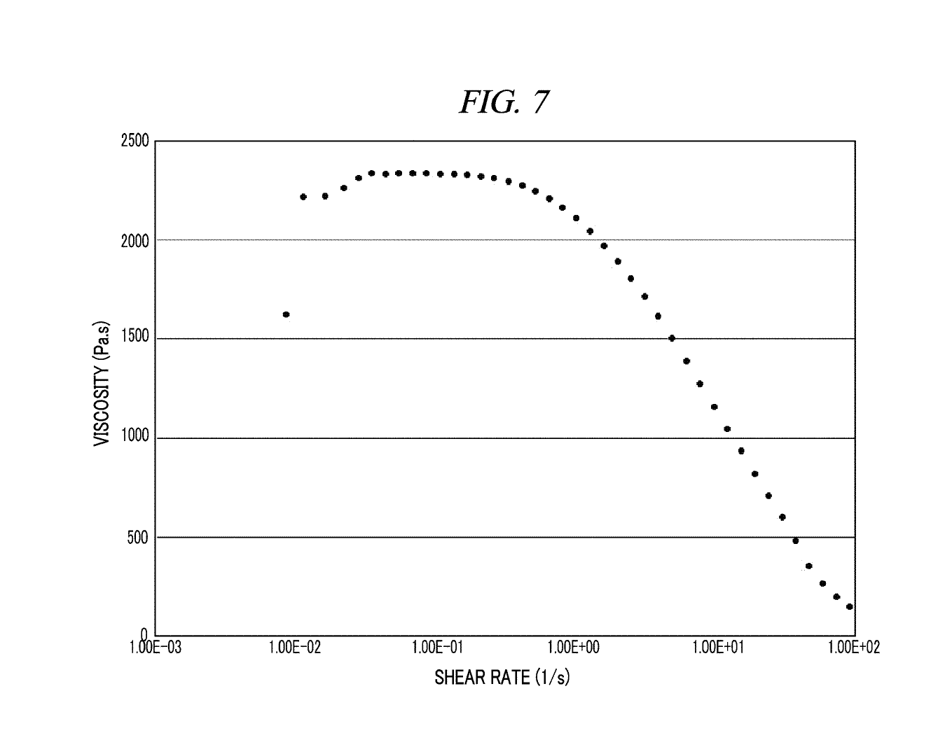

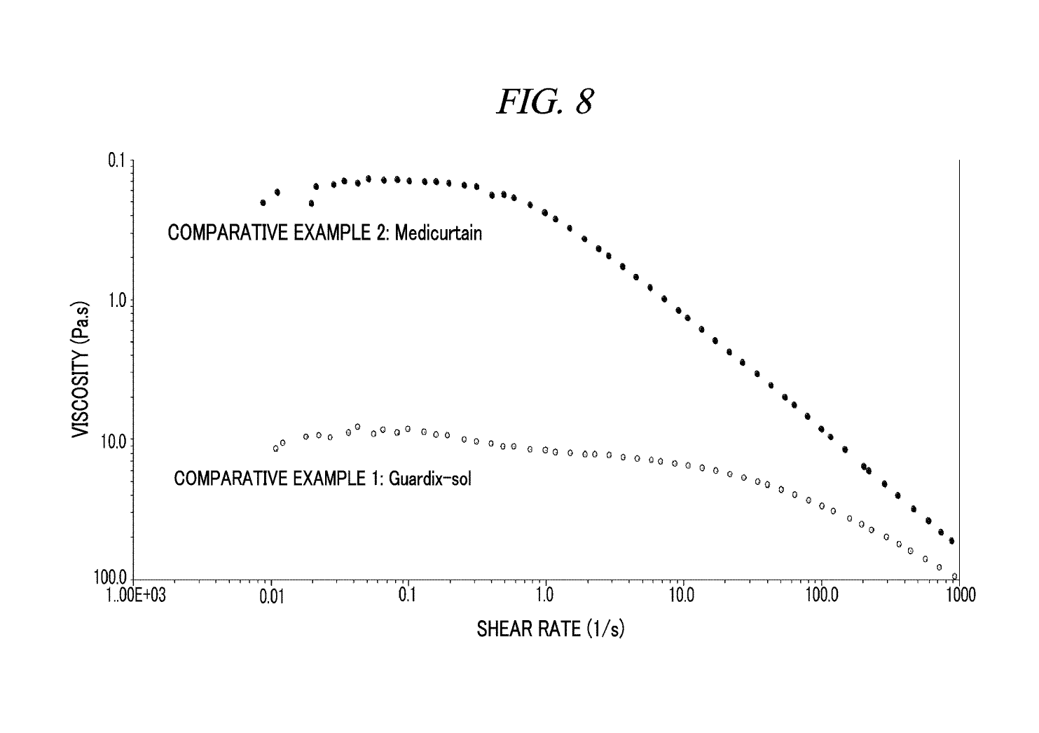

FIG. 7 shows a result of evaluation of the viscosity of the adhesion-preventing hydrogel in accordance with Example 1 of the present disclosure, and FIG. 8 shows a result of evaluation of the viscosity of the adhesion-preventing hydrogels as the products of other companies (Comparative Example 1: Medicurtain, Comparative Example 2: Guardix-sol). As shown in FIG. 7, the adhesion-preventing hydrogel according to Example 1 has a viscosity of from 1300 pascal·sec to 1400 pascal·sec determined at a temperature of 25° C. and a shear rate of 1 sec⁻¹, and, thus, it was confirmed that the adhesion-preventing hydrogel according to Example 1 has a viscosity 10 times to 15 times higher than that of the existing commercial hydrogel products for adhesion prevention according to Comparative Examples 1 and 2 as shown in FIG. 8.

As a result, it was confirmed that the adhesion-preventing hydrogel prepared according to an example of the present disclosure has a high viscoelasticity and a high viscosity.

### 2. Evaluation of Flowability of Adhesion-Preventing Hydrogel

The adhesion-preventing hydrogel according to Example 1 was prepared and the adhesion-preventing agents according to Comparative Examples 1 to 3 as control groups of the adhesion-preventing hydrogel of Example 1 were selected from the products of the first company, the second company, and the third company and prepared (Comparative Example 1: Medicurtain, Comparative Example 2: Guardix-sol, and Comparative Example 3: Protescal).

The adhesion-preventing hydrogel according to Example 1 and the adhesion-preventing agents according to Comparative Examples 1 to 3 were dropped onto the abdominal muscles collected from laboratory rats to a predetermined size and fixed to a flat plate, and the adhesion-preventing hydrogel according to Example 1 and the adhesion-preventing agents according to Comparative Examples 1 to 3 were compared in terms of flowability by vertically standing the plates fixing the abdominal muscles of the rats onto which the adhesion-preventing hydrogel according to Example 1 and the adhesion-preventing agents according to Comparative Examples 1 to 3 were dropped.

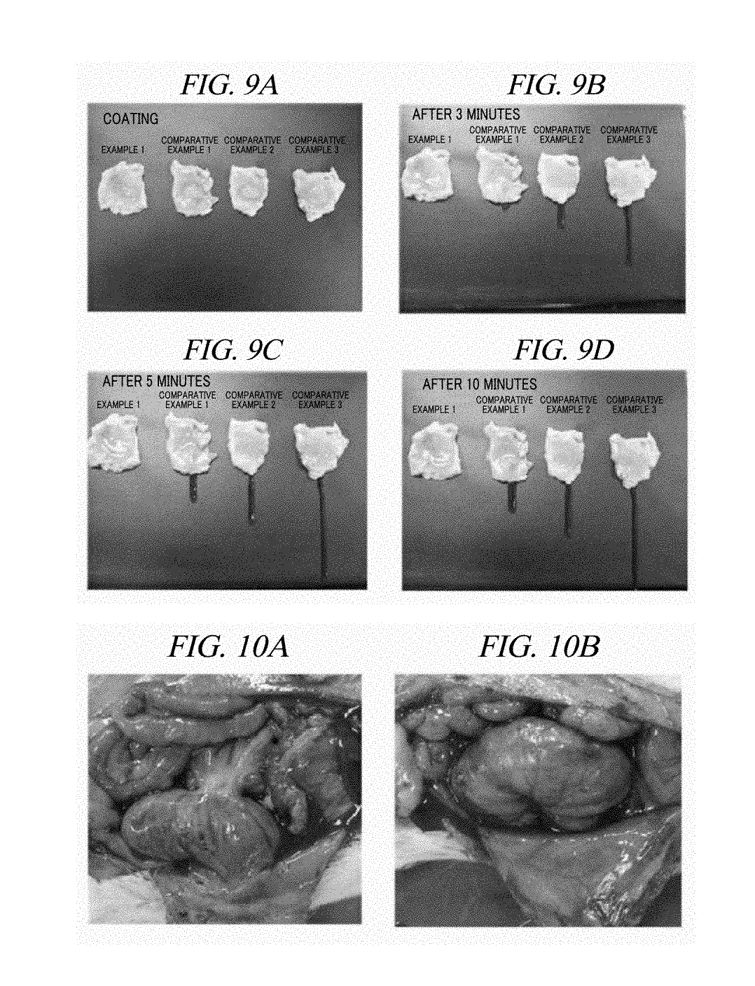

FIG. 9 shows a result of evaluation of the flowability of the adhesion-preventing hydrogel in accordance with Example 1 of the present disclosure and the adhesion-preventing hydrogels in accordance with Comparative Examples 1 to 3. As shown in FIG. 9, it was confirmed that the adhesion-preventing hydrogel according to Example 1 did not flow from an area where it was dropped even after some time from the dropping, whereas the adhesion-preventing agents according to Comparative Examples 1 to 3 continuously flowed from the respective areas where they were dropped with the lapse of time from the dropping.

Therefore, it was confirmed that the adhesion-preventing hydrogel according to Example 1 has an excellent shape-keeping performance and thus does not flow easily as compared with the existing commercial adhesion-preventing agents according to Comparative Examples 1 to 3.

### 3. Evaluation of Adhesion Prevention of Adhesion-Preventing Hydrogel

The adhesion-preventing hydrogels according to Examples 1 to 4 and a saline solution according to Comparative Example 4 as a control group of the adhesion-preventing hydrogels according to Examples 1 to 4 were prepared, and the adhesion-preventing hydrogels according to Examples 1 to 4 and the saline solution according to Comparative Example 4 were applied to the damaged appendices and abdominal abrasion models of laboratory rats [7-week-old male Sprague-Dawley rats (SLC)] to perform the evaluation of adhesion prevention. In order to induce adhesion in the respective laboratory rats to be applied with the adhesion-preventing hydrogels according to Examples 1 to 4 and the saline solution according to Comparative Example 4, the laboratory rats' abdomen was cut open to take out the appendix and the appendix was rubbed to form a damaged surface having a size of 1.2 cm×1.2. cm, and the rats' abdominal cavity membrane positioned corresponding to the damaged surface of the appendix within the abdomen of the rats was damaged by the above-described method to form a damaged surface having the same size as the damaged surface of the appendix.

Then, the appendix was inserted into the abdomen of the laboratory rats and the damaged surface of the appendix and the damaged surface of the abdominal cavity membrane were brought into contact with each other and sutured. Then, the adhesion-preventing hydrogels according to Examples 1 to 4 and the saline solution according to Comparative Example 4 were coated on the damaged surface of each laboratory rat where the appendix and the abdominal cavity membrane face each other and the cut-open abdomen of the laboratory rats was sutured again. After suture, the respective rats were fed with sufficient water and food and grown for one week and then victimized to perform the evaluation of adhesion prevention. The result thereof was as shown in the following Table 3.

TABLE 3

| | | | | Adhesion area |
|---|---|---|---|---|
| | Degree of | Strength of | Adhesion area | reduction ratio |
| | adhesion | adhesion | (cm²) | (%) |
| Control | 3.75 ± 0.46* | 3.05 ± 0.00* | 0.84 ± 0.00* | 0 |
| Group | | | | |
| Example 1 | 0.00 ± 0.0** | 0.00 ± 0.0** | 0.00 ± 0.0** | 100 |
| Example 2 | 0.00 ± 0.0** | 0.00 ± 0.0** | 0.00 ± 0.0** | 100 |
| Example 3 | 0.00 ± 0.0** | 0.00 ± 0.0** | 0.00 ± 0.0** | 100 |
| Example 4 | 0.00 ± 0.0** | 0.00 ± 0.0** | 0.00 ± 0.0** | 100 |

The degree of adhesion shown in Table 3 was evaluated from 0 to 5. The data shown in Table 3 represent mean±S.D (n=0.5), and * indicates the significance p<0.05 compared with the control group and ** indicates the significance p<0.05 compared with the control group. The range of value (from 0 to 5) for evaluating the degree of adhesion was determined depending on the following states: no adhesion (0); single thin film-type adhesion (1); two or more thin film-type adhesions (2); point-type concentrated thick adhesion (3); plate-type concentrated adhesion (4); and very thick adhesion having blood vessels therein or one or more plate-type thick adhesion (5).

Further, the strength of adhesion shown in Table 3 was evaluated from 1 to 4. The range of value (from 1 to 4) for evaluating the strength of adhesion was determined depending on the following states: film-type adhesion which can be detached with very weak force (1); adhesion which can be detached with moderate force (2); adhesion which can be detached with force of significant pressure (3); and adhesion which is difficult to detach or can be detached with force of very high pressure (4). As shown in Table 3, it was confirmed that in the damaged organ coated with Comparative Example 4 using the saline solution as an adhesion-preventing agent, adhesion which is close to the plate-type concentrated adhesion and can be detached with force of significant pressure occurred and in the damaged organs coated with the adhesion-preventing hydrogels according to Examples 1 to 4, adhesion did not occur. Herein, it can be seen that the adhesion-preventing hydrogels according to Examples 1 to 4 did not cause the occurrence of adhesion regardless of whether the sodium hyaluronate which can be a material of the adhesion-preventing hydrogels has a high molecular weight (Examples 1 and 2) or a low molecular weight (Examples 3 and 4). That is, the adhesion-preventing hydrogels are not greatly affected by a molecular weight of a hyaluronic acid substrate which can be contained as a material, and, thus, it is possible to more flexibly select materials.

FIG. 10 shows a result of evaluation of adhesion prevention of the adhesion-preventing hydrogel in accordance with Example 1 of the present disclosure. The adhesion prevention using the adhesion-preventing hydrogel according to Example 1 of the present disclosure can be visually seen from the result of adhesion prevention in the damaged surfaces of the laboratory rat's appendix and abdominal cavity membrane coated with the adhesion-preventing hydrogel according to Example 1 in FIG. 10. As shown in FIG. 10, it can be seen that adhesion occurred in the damaged surfaces of the laboratory rat's appendix and abdominal cavity membrane coated with the saline solution according to Comparative Example 4 (FIG. 10A) but did not occur in the damaged surfaces of the laboratory rat's appendix and abdominal cavity membrane coated with the adhesion-preventing hydrogel according to Example 1 (FIG. 10B).

Therefore, it was confirmed that an adhesion-preventing hydrogel according to an example of the present disclosure acts on a damaged organ without deviation from a coating surface on the damaged organ even after some time from coating and thus is very effective in preventing adhesion.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF SYMBOLS

1: hyaluronic acid substrate
2: poly-L-lysine
3: coagulated precipitates
4: hydrogel
s: solvent
d: dispersion solvent
10: container
20: double boiler
30: autoclave

We claim:

1. An adhesion-preventing hydrogel, comprising a composite of a hyaluronic acid substrate with epsilon poly-L-lysine,
wherein the composite is prepared by heat-treating a suspension containing coagulated precipitates obtained from a mixed solution of the hyaluronic acid substrate with the epsilon poly-L-lysine.

2. The adhesion-preventing hydrogel of claim 1,
wherein the heat-treating is performed at a temperature ranging from 50° C. to 150° C.

3. The adhesion-preventing hydrogel of claim 1,
wherein the hyaluronic acid substrate includes a hyaluronic acid or a salt of the hyaluronic acid.

4. The adhesion-preventing hydrogel of claim 3,
wherein the salt of the hyaluronic acid includes a material selected from the group consisting of sodium hyaluronate, potassium hyaluronate, calcium hyaluronate, magnesium hyaluronate, zinc hyaluronate, cobalt hyaluronate, tetrabutylammonium hyaluronate, and combinations thereof.

5. The adhesion-preventing hydrogel of claim 1,
wherein a weight ratio of the hyaluronic acid substrate to the epsilon poly-L-lysine is from 1: 0.2 to 4.

6. The adhesion-preventing hydrogel of claim 1,
wherein a molecular weight of the hyaluronic acid substrate is from 10,000 Daltons to 5,000,000 Daltons, and a molecular weight of the epsilon poly-L-Lysine is from 3,000 Daltons to 10,000 Daltons.

7. A method of preparing an adhesion-preventing hydrogel, comprising:
preparing a mixed solution by mixing a solution containing a hyaluronic acid substrate with a solution containing epsilon poly-L-lysine;
separating coagulated precipitates of the hyaluronic acid substrate and the epsilon poly-L-lysine formed in the mixed solution;
dispersing the separated coagulated precipitates in a dispersion solvent to prepare a suspension; and
heat-treating the suspension to obtain an adhesion-preventing hydrogel including a composite of the hyaluronic acid substrate with the epsilon poly-L-lysine.

8. The method of preparing an adhesion-preventing hydrogel of claim 7,
wherein a molecular weight of the hyaluronic acid substrate is from 10,000 Daltons to 5,000,000 Daltons, and a molecular weight of the epsilon poly-L-Lysine is from 3,000 Daltons to 10,000 Daltons.

9. The method of preparing an adhesion-preventing hydrogel of claim 7,
wherein the hyaluronic acid substrate includes hyaluronic acid or a salt of the hyaluronic acid.

10. The method of preparing an adhesion-preventing hydrogel of claim 7,
wherein the salt of the hyaluronic acid includes a material selected from the group consisting of sodium hyaluronate, potassium hyaluronate, calcium hyaluronate, magnesium hyaluronate, zinc hyaluronate, cobalt hyaluronate, tetrabutylammonium hyaluronate, and combinations thereof.

11. The method of preparing an adhesion-preventing hydrogel of claim 7,
wherein the heat-treating of the suspension is performed by double-boiling the suspension at a temperature ranging from 50° C. to 150° C. for 40 to 80 minutes.

12. The method of preparing an adhesion-preventing hydrogel of claim 7,
wherein the adhesion-preventing hydrogel is obtained by heat-treating the suspension using an autoclave at a temperature ranging from 50° C. to 150° for 5 to 30 minutes.

13. The method of preparing an adhesion-preventing hydrogel of claim 7,
wherein a weight ratio of the hyaluronic acid substrate to the epsilon poly-L-lysine in the mixed solution is from 1: 0.2 to 4.
